(19) United States
(12) Patent Application Publication
(10) Pub. No.: US 2026/0199016 A1
(43) Pub. Date: Jul. 16, 2026

(54) Systems and Methods Associated with Selection and Deployment of Neurovascular Devices

(71) Applicants: Stryker Corporation, Portage, MI (US); Stryker European Operations Limited, Cork (IE)

(72) Inventors: Lakshmanan Chettiar, Gurgaon (IN); Sreekanth Padmanabhan, Gurgaon (IN); Venkateswaran Perumal, Bangalore (IN); Jayendiran Raja, Chennai (IN)

(21) Appl. No.: 19/138,201

(22) PCT Filed: Jan. 11, 2024
(86) PCT No.: PCT/US2024/011191

§ 371 Date: Jun. 12, 2025

Related U.S. Application Data
(60) Provisional application No. 63/480,385, filed on Jan. 18, 2023.

(51) Int. Cl. A61B 34/10; A61B 6/50; A61F 2/82; A61F 2/90; G16H 50/50

(52) U.S. Cl. CPC A61B 34/10; A61F 2/90; G16H 50/50; A61B 6/504; A61B 2034/104; A61B 2034/105; A61B 2034/108; A61F 2002/823

(57) ABSTRACT

An example method includes: receiving images of a vessel having an aneurysm captured by an image-capture device; reconstructing using the images, the vessel to generate a model of the vessel; determining taking foreshortening effect into consideration, a final length of a stent when the stent is deployed inside the model of the vessel to divert flow from the aneury sm; visually presenting a display of the stent deployed within the model of the vessel; and providing parameters of the stent including apposition and pore density of the stent.

### CROSS REFERENCE TO RELATED APPLICATION

The present application claims priority to U.S. Provisional Patent Application No. 63/480,385, filed on Jan. 18, 2023, the entire contents of which are herein incorporated by reference as if fully set forth in this description.

### BACKGROUND

An aneurysm occurs when part of an artery wall weakens, allowing it to abnormally balloon out or widen. Aneurysms often occur in the aorta, brain, back of the knee, intestine, or spleen. A ruptured aneurysm can cause internal bleeding, which may lead to a stroke, and can sometimes be fatal.

Aneurysms often have no symptoms until they rupture. Treatment of aneurysms varies from watchful waiting to emergency surgery. The choice of treatment depends on the location, size, and condition of the aneurysm.

Some aneurysms may require surgery to reinforce the artery wall with a stent or coil. When the aneurysm has ballooned out from the side of the blood vessel, a clip or coiling procedure may close off the area.

Currently, healthcare professionals (e.g., neurosurgeons or interventional radiologists) may obtain images of the vessel and aneurysm, try to estimate the dimensions of the aneurysm (e.g., diameter) and then try to determine based on their experience what type of neurovascular device (e.g., stent or coil) is appropriate for the aneurysm and the parameters (e.g., diameter, length, etc.) of such device.

However, two-dimensional (2D) images of a three-dimensional (3D) environment can be deceiving, and the estimates by the healthcare professional of the size and characteristics of the aneurysm and the vessel in which the aneurysm has formed might not be accurate. Based on such inaccurate estimates, the healthcare professional may select a device with certain parameters and deploy it in the vessel. However, due to inaccuracies in the estimates, the device may be oversized, undersized, or might not be proper for the aneurysm. The healthcare professional may then try a different device with different parameters.

As such, the healthcare professional may employ a trial and error approach to determine the type of device to use and its key parameters. This approach is not optimal, may reduce the efficacy of the device when deployed, and may increase the risk of rupture of the aneurysm.

It is with respect to these and other considerations that the disclosure made herein is presented.

### SUMMARY

Within examples, described herein are systems and methods associated with selection and deployment of neurovascular devices.

Within additional examples described herein, systems and methods are disclosed that relate to capturing images of an aneurysm in a blood vessel of a patient, using the images to determine what type of a neurovascular device is optimal for that aneurysm and the key parameters of such a neurovascular device, and visually presenting the neurovascular device inside the vessel while presenting the key parameters to the healthcare professional.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying Figures.

FIG. 1 illustrates a brain aneurysm, according to an example.

FIG. 2 illustrates dimensions and ratios related to an aneurysm, according to an example.

FIG. 3 is a block diagram of a system including an image-capture device, a neurovascular module, and a display device, according to an example implementation.

FIG. 4 is a flowchart of a method for determining and providing key parameters of a stent, according to an example implementation.

FIG. 5 illustrates a reconstructed vessel generated by the neurovascular module of FIG. 4, according to an example implementation.

FIG. 6 is a flowchart of a method for determining, taking foreshortening effect into consideration, a final length of a stent when the sent is deployed inside a reconstructed vessel, according to an example implementation.

FIG. 7 illustrates a stent deployed within the reconstructed vessel of FIG. 5, according to an example implementation.

FIG. 8 illustrates a cross-sectional view of a stent deployed within a vessel having an aneurysm, according to an example implementation.

FIG. 9 illustrates a graph showing variation of apposition of a stent along aa length of a vessel, according to an example implementation.

FIG. 10 illustrates a graphical user interface, according to an example implementation.

FIG. 11 illustrates a micro computerized tomography scan of a vessel having an aneurysm and a stent exhibiting ribboning.

FIG. 12 illustrates a reconstructed vessel with an aneurysm and a region where

ribboning is most likely to occur, according to an example implementation.

FIG. 13 illustrates a block diagram of a system for flow visualization and quantification, according to an example implementation.

FIG. 14 illustrates different geometric characteristics of an aneurysm bulging from a vessel, according to an example implementation.

FIG. 15 illustrates blood flow through a vessel and aneurysm before treatment, according to an example implementation.

FIG. 16A illustrates blood flow through the vessel and the aneurysm of FIG. 15 after treatment, according to an example implementation.

FIG. 16B illustrates pore area of the FD device of FIG. 16A, according to an example implementation.

FIG. 17 illustrates is a block diagram of a computing device, according to an example implementation.

FIG. 18 is a flowchart of a method for selecting a neurovascular device and providing key parameters of the neurovascular device, according to an example implementation.

FIG. 19 is a flowchart of additional operations that are executable with the method of FIG. 18, according to an example implementation.

FIG. 20 is a flowchart of additional operations that are executable with the method of FIG. 18, according to an example implementation.

FIG. 21 is a flowchart of additional operations that are executable with the method of FIG. 18, according to an example implementation.

FIG. 22 is a flowchart of additional operations that are executable with the method of FIG. 18, according to an example implementation.

FIG. 23 is a flowchart of additional operations that are executable with the method of FIG. 18, according to an example implementation.

FIG. 24 is a flowchart of additional operations that are executable with the method of FIG. 18, according to an example implementation.

FIG. 25 is a flowchart of additional operations that are executable with the method of FIG. 18, according to an example implementation.

FIG. 26 is a flowchart of additional operations that are executable with the method of FIG. 18, according to an example implementation.

### DETAILED DESCRIPTION

Implementations described herein are relevant to improving selection and deployment of a neurovascular device in a vessel having an aneurysm. An image-capture device captures images of the vessel and the aneurysm and provides the images to a neurovascular module. The neurovascular module reconstructs the geometry (e.g., determines a three-dimensional model) of the vessel and aneurysm from the images. The neurovascular module then determines what type of neurovascular device (e.g., a braided stent, a laser-cut stent, a coil, etc.) is suitable or optimal for the particular aneurysm.

The neurovascular module also determines the key parameters of the selected device. For example, for a braided stent, the neurovascular module may determine one or more key parameters selected from among a group of parameters including size (e.g., length and diameter), vessel wall apposition, pore density variation, etc. of the stent. In determining such key parameters, the neurovascular module takes into consideration effects such as foreshortening effects that might cause conventional approaches to fail in selecting the appropriate stent.

In an example, the neurovascular module may further predict locations within the vessel where ribboning may occur. This may provide the healthcare professional with information that helps optimize technique of deployment of the stent to avoid ribboning.

The neurovascular module may also be in communication with a display device. The neurovascular module may visually present on the display device a visualization of the stent deployed within the vessel. The neurovascular module may also generate a display of the key parameters of the stent. As such, the healthcare professional may reduce or avoid trial and error, and may be enabled to preplan deployment of a neurovascular device in an enhanced manner.

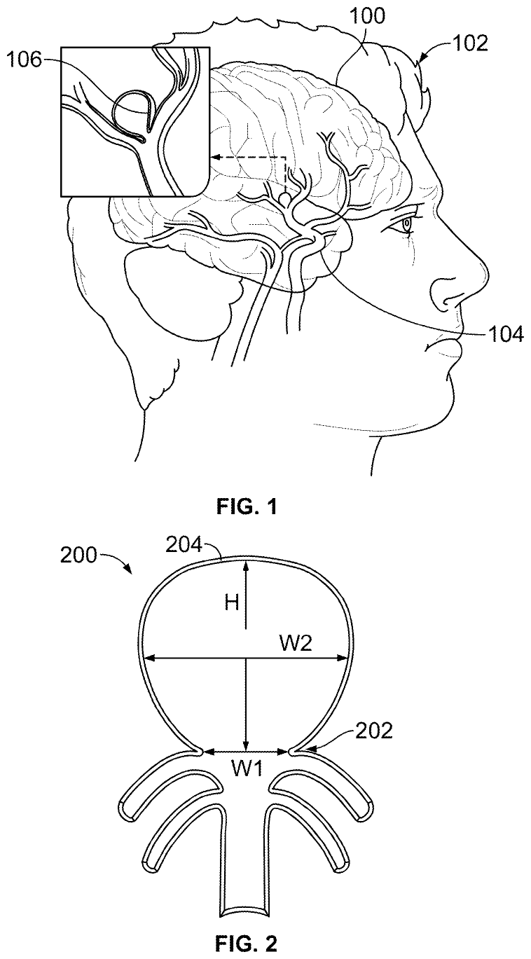

FIG. 1 illustrates a brain aneurysm, according to an example. A brain aneurysm is used herein as an example for illustration. The systems and methods described herein can be used with an aneurysm in any blood vessel.

In the illustrated example, a brain 100 of a patient 102 has several blood vessel such as artery 104. The artery 104 is a muscular-walled tube blood vessel that is a part of the blood circulation system of the patient 102. The artery 104 conveys blood from the heart to the brain 100, carrying oxygen and nutrients to support the brain 100 and its functions.

A diagnosis of a brain aneurysm in the artery 104 indicates that a bulging, weak area exists in the wall of one of the arteries that supplies blood to the brain. As depicted in FIG. 1, a cerebral aneurysm 106 may form in the artery 104. As mentioned above, a brain or cerebral aneurysm is used herein as an example. Other types of aneurysms includes aortic aneurysm, popliteal artery aneurysm, mesenteric artery aneurysm, and splenic artery aneurysm, for example.

In some cases, it may be desirable to insert or deploy a neurovascular device such as a stent (e.g., a braided stent or laser-cut stent) or a coil in the artery 104 or the cerebral aneurysm 106 to divert blood flow away from the cerebral aneurysm 106 and prevent it from rupturing. The type of device to use is based on the characteristics (e.g., dimensions and configuration) of the cerebral aneurysm 106.

FIG. 2 illustrates dimensions and ratios related to an aneurysm 200, according to an example. The aneurysm 200 may represent the cerebral aneurysm 106, for example.

The aneurysm 200 has a neck 202 (a narrowing portion where the aneurysm 200 emanates from the vessel) and a dome 204. A size of the aneurysm 200 may be characterized by several dimensions such as neck width “W1,” dome width “W2,” and dome height “H.” The size can also be characterized by ratios of these dimensions such as

$$Aspect Ratio = \frac{H}{{W}_{1}} andDome / Neck Ratio = \frac{{W}_{2}}{{W}_{1}} .$$

These dimensions and ratios may help determining which type of neurovascular device to use. The characteristics and dimensions of the vessel from which the aneurysm 200 bulges may also facilitate determining the key parameters of the neurovascular device. The systems disclosed herein are configured to (i) capture images of a vessel and an aneurysm, (ii) determine, based on the captured images, characteristics of the aneurysm and the vessel, (iii) accordingly determine, based on the determined characteristics, the type of neurovascular device to use and the key parameters of the neurovascular device, and (iv) visually present the vessel and aneurysm, the neurovascular device deployed in the vessel, and the key parameters of the neurovascular device to a healthcare professional.

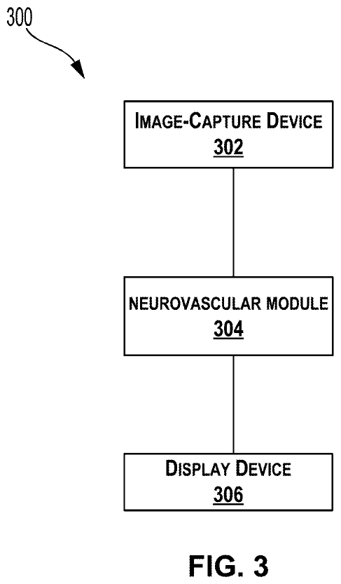

FIG. 3 is a block diagram of a system 300 including an image-capture device 302, a neurovascular module 304, and a display device 306, according to an example implementation. Components of the system 300 may be configured to work in an interconnected fashion with each other and/or with other components coupled to respective systems. One or more of the described operations or components of the system 300 may be divided up into additional operational or physical components, or combined into fewer operational or physical components. In some further examples, additional operational and/or physical components may be added to the system 300. Still further, any of the components or modules of the system 300 may include or be provided in the form of a processor (e.g., a microprocessor, a digital signal processor, etc.) configured to execute program code including one or more instructions for implementing logical operations described herein.

The system 300 may further include any type of computer readable medium (non-transitory medium) or memory, for example, such as a storage device including a disk or hard drive, to store the program code that when executed by one or more processors cause the system 300 to perform the operations described above. In an example, the system 300 may be included within other systems.

The image-capture device 302 is configured to capture images of the vessel having the aneurysm. For example, the image-capture device 302 can include a computerized tomography (CT) scanning device that combines a series of X-ray images taken from different angles around a body of the patient having the aneurysm and uses computer processing to generate cross-sectional images (slices) of the blood vessels.

In particular, the image-capture device 302 can include a micro-CT scanning device that uses a 3D imaging technique utilizing X-rays to see inside the body of the patient, slice by slice. Micro-CT scanning is similar to CT scan imaging but on a small scale with enhanced resolution. For example, vessels and aneurysms can be imaged with pixel sizes as small as 100 nanometers and objects can be scanned as large as 200 millimeters in diameter. Thus, micro-CT imaging can be suitable for capturing images of an aneurysm.

As such, in an example, the image-capture device 302 can include an X-ray source generating X-rays that are then transmitted through the part of the patient that has the aneurysm. The image-capture device 302 also includes an X-ray detector that records the X-rays as a 2D projection image. The X-ray source may then be rotated a fraction of a degree on a rotational platform, and another X-ray projection image is taken. This step is repeated through a 180-degree or 360 degrees, thereby capturing images of the aneurysm from different angles.

The neurovascular module 304 is configured to receive such series of X-ray projection images, and is then configured to generate cross-sectional images through a computational process that can be referred to as “reconstruction.” For example, the neurovascular module 304 can use the images to generate 3D models of the vessel and the aneurysm.

The neurovascular module 304 is configured to then analyze the “slices” of cross-sectional images and/or models to extract characteristics of the aneurysm (e.g., dimensions and ratios of the aneurysm) and the blood vessel from which the aneurysm bulges. Based on such determination, the neurovascular module 304 is configured to determine a suitable neurovascular device (e.g., a coil, a braided stent, a laser-cut stent, etc.).

The neurovascular module 304 is further configured to provide key parameters of the neurovascular device. For example, if the neurovascular device is selected to be a braided stent, the neurovascular module 304 may provide parameters including the size (e.g., diameter and length) of the stent, expected apposition, pore density, etc. In determining the key parameters of the stent, the neurovascular module 304 takes into consideration foreshortening effects as described in more details below.

The neurovascular module 304 communicates such information to the display device 306, which visually presents the information to the healthcare professional. The healthcare professional may then select a commercially available stent that matches the key parameters provided by the neurovascular module 304. Operations performed by the neurovascular module 304 to determine the key parameters of a stent are described next.

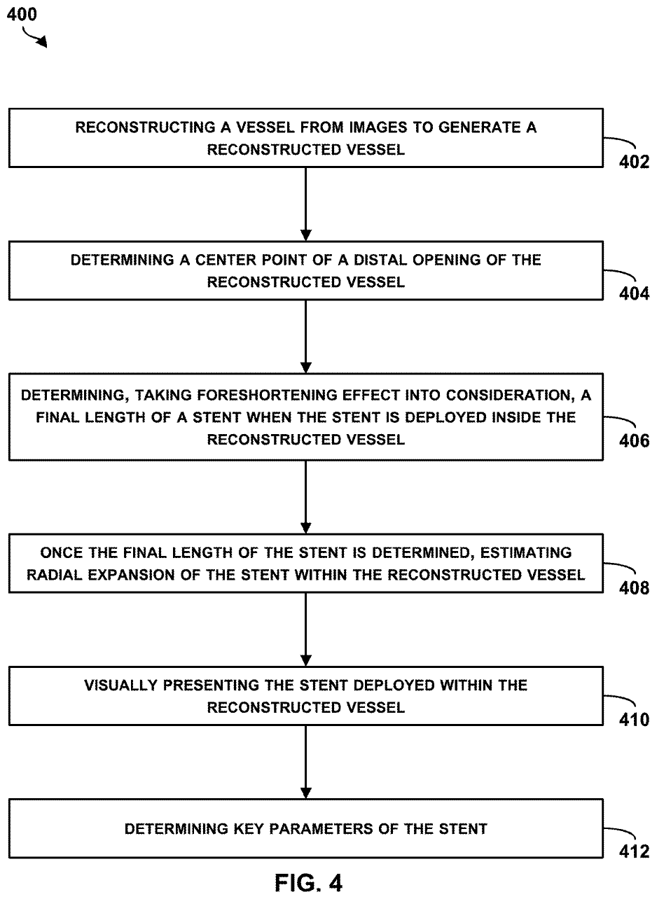

FIG. 4 is a flowchart of a method 400 for determining and providing key parameters of a stent, according to an example implementation. The method 400 can, for example, be performed by the neurovascular module 304.

The method 400 may include one or more operations, or actions as illustrated by one or more of blocks 402-412 (and associated blocks 602-612 of the method 600 described below). Although the blocks are illustrated in a sequential order, these blocks may in some instances be performed in parallel, and/or in a different order than those described herein. Also, the various blocks may be combined into fewer blocks, divided into additional blocks, and/or removed based upon the desired implementation.

In addition, for the method 400 and other processes and operations disclosed herein, the flowchart shows operation of one possible implementation of present examples. In this regard, each block may represent a module, a segment, or a portion of program code, which includes one or more instructions executable by processors for implementing specific logical operations or steps in the process. The program code may be stored on any type of computer readable medium or memory, for example, such as a storage device including a disk or hard drive. The computer readable medium may include a non-transitory computer readable medium or memory, for example, such as computer-readable media that stores data for short periods of time like register memory, processor cache and Random Access Memory (RAM). The computer readable medium may also include non-transitory media or memory, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, a tangible storage device, or other article of manufacture, for example. In addition, for the method 400 and other processes and operations disclosed herein, one or more blocks in FIG. 4 may represent circuitry or digital logic that is arranged to perform the specific logical operations in the process.

At block 402, the method 400 includes reconstructing a vessel from images to generate a reconstructed vessel (e.g., a model of the vessel such as a 3D model). The images could be received at the neurovascular module 304 from the image-capture device 302 as described above, and the neurovascular module 304 can generate a 3D mesh of at least a portion of the vessel that includes the aneurysm, for example.

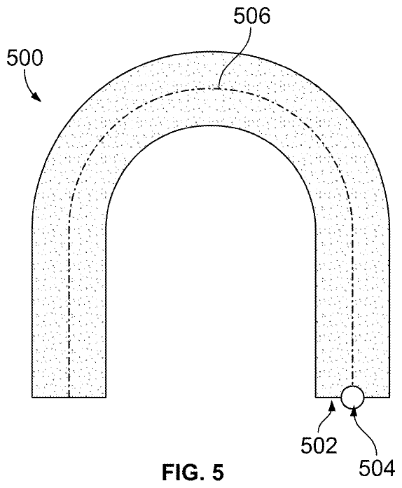

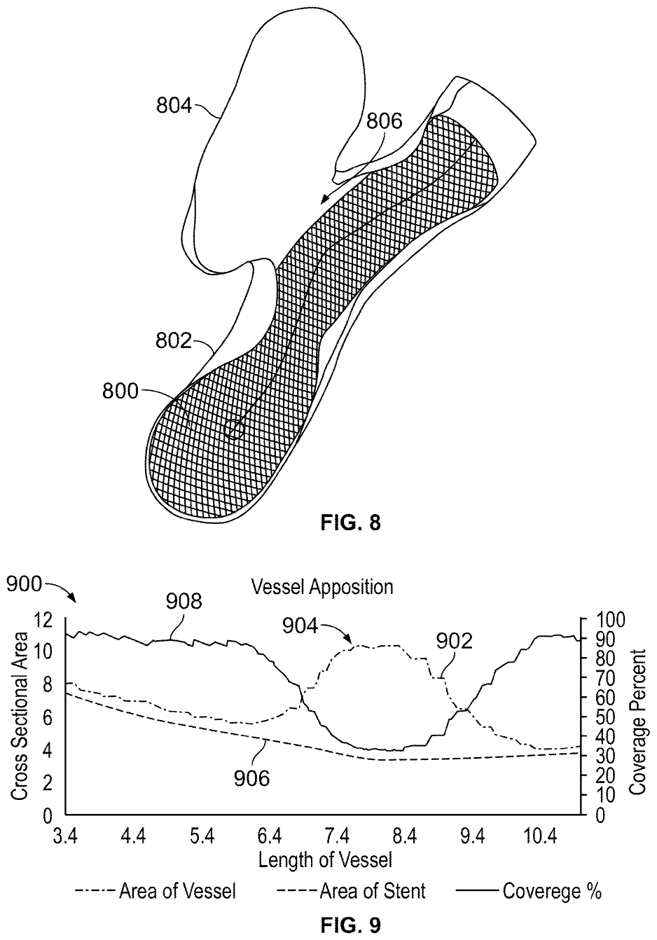

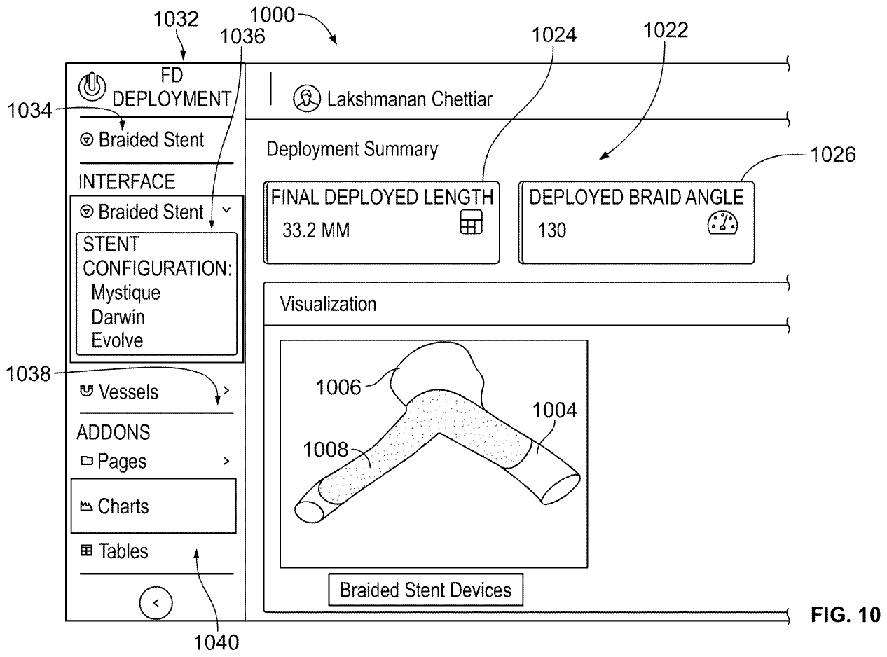

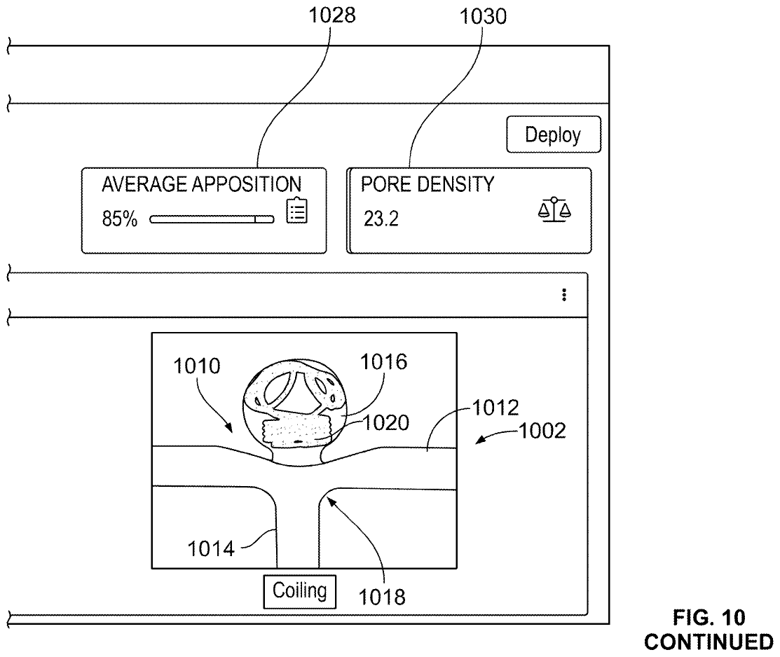

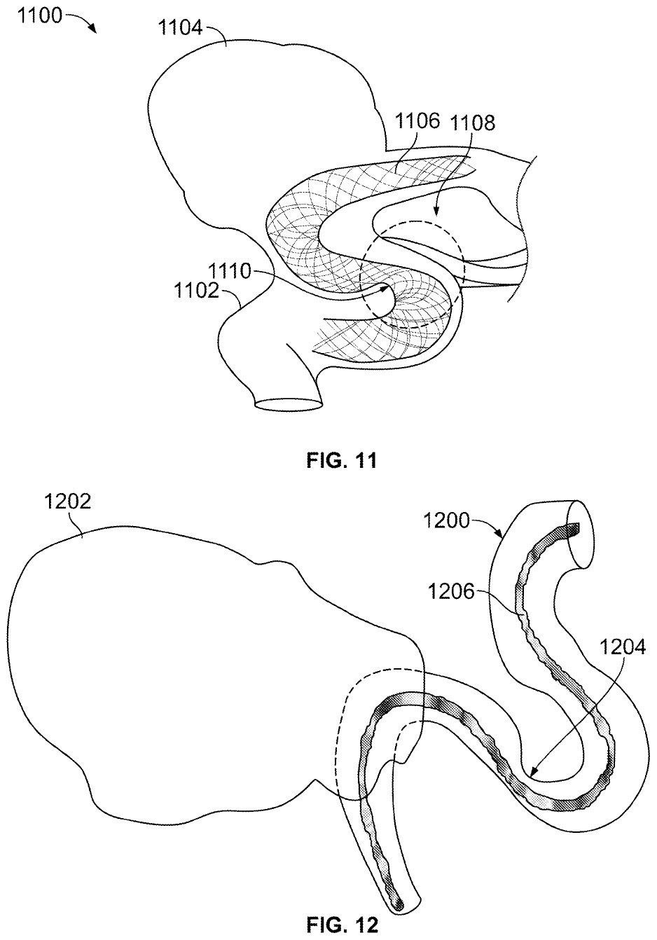

FIG. 5 illustrates a reconstructed vessel 500 generated by the neurovascular module 304, according to an example implementation. The reconstructed vessel 500 is depicted as a yoke-shaped (e.g., U-shaped) vessel for simplicity. Actual blood vessel might have complex shapes as shown in FIG. 8, FIG. 10, and FIG. 11, for example.

Referring to FIG. 4, at block 404, the method 400 includes determining a center point of a distal opening of the reconstructed vessel. As depicted in FIG. 5, the neurovascular module 304 can determine a distal opening 502 of the reconstructed vessel 500. The neurovascular module 304 can then determine a center point 504 of the distal opening 502. For example, the neurovascular module 304 can determine a point on a plane of the distal opening 502, wherein such point is disposed at a given distance from substantially all points on a perimeter of the distal opening 502. The neurovascular module 304 then designates such point as the center point 504.

Referring to FIG. 4, at block 406, the method 400 includes determining, taking foreshortening effect into consideration, a final length of a stent when the sent is deployed inside the reconstructed vessel. A braided stent is advantageously highly maneuverable, allowing a healthcare professional (e.g., a surgeon or interventional radiologist) to reach distal regions within the intracranial vasculature for their deployment. The stent operates as a flow diverter for aneurysm occlusion. The stent operating as a flow diverter is used to change hemodynamic conditions in the vicinity of the aneurysm, redirecting blood flow away from the aneurysm into the parent vessel from which the aneurysm bulges, thus promoting controlled thrombosis inside the aneurysm sac and restoring normal blood flow.

The braided stent may have a dense mesh of interwoven wires (e.g., 24 wires or more). As an example, such stent can be made if a cobalt-chromium metal alloy characterized with high specific strength.

A technical problem that occurs when using a braided stent in neurovascular procedures is the difficulty of predicting the final positioning of the stent after deployment inside the vessel due to the change in length of the braided stent, which is dependent on the anatomy of the patient and the positioning of the device within it. In other words, an issue of deployment of braided stents is the change in total length (foreshortening) that the stent experiences when the stent is released in the blood vessel from a catheter. The method 400 thus includes taking foreshortening into consideration when determining the final length and position of the stent once deployed. Determining the final length may involve several operations.

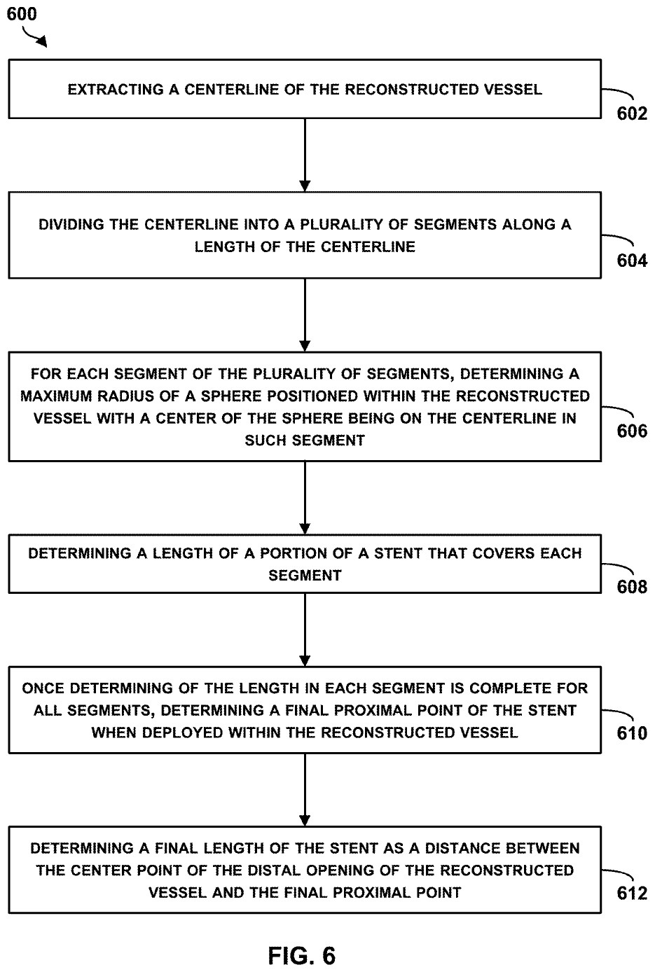

FIG. 6 is a flowchart of a method 600 for determining, taking foreshortening effect into consideration, a final length of a stent when the sent is deployed inside the reconstructed vessel 500, in accordance with an example for illustration. The method 600 can be performed by the neurovascular module 304 to execute the operation of the block 406, for example.

At block 602, the method 600 includes extracting a centerline of the reconstructed vessel 500. Referring to FIG. 5, the neurovascular module 304 can extract a centerline 506 along a length of the reconstructed vessel 500. For example, the neurovascular module 304 can divide the reconstructed vessel 500 into multiple segments or cross-sections along a length of the reconstructed vessel 500. The neurovascular module 304 can then determine a center point of the reconstructed vessel 500 at each segment of the multiple segments, taking into consideration curvature and tortuousness of the reconstructed vessel 500. The neurovascular module 304 can then extrapolate or connect such center points of the segments to determine or extract the centerline 506 of the reconstructed vessel 500.

At block 604, the method 600 includes dividing the centerline 506 into a plurality of segments along a length of the centerline 506. The number of segments can vary. As an example, the neurovascular module 304 can divide the centerline 506 into 5000 or 10,000 segments depending on the length of the reconstructed vessel 500.

At block 606, the method 600 includes, for each segment of the plurality of segments, determining a maximum radius of a sphere positioned within the reconstructed vessel 500 with a center of the sphere being on the centerline 506 in such segment. As an example for illustration, the neurovascular module 304 can implement a k-nearest neighbors algorithm (KNN) to determine the radius of the sphere. KNN is a non-parametric, supervised learning classifier, which uses proximity to make classifications or predictions about the grouping of an individual data point. Particularly, the KNN algorithm can determine a radius from a given point on the centerline 506 within a segment of the plurality of segments and form a sphere centered at the given point such that the sphere contacts the inner walls of the reconstructed vessel 500.

This way, the neurovascular module 304 can estimate or determine the radius of the reconstructed vessel 500 at each segment of the plurality of segments along a length of the centerline 506. As mentioned above, the reconstructed vessel 500 depicted in FIG. 5 is a simplified configuration wherein the reconstructed vessel 500 has a consistent radius throughout its length. However, an actual vessel of a patient is likely to have a variable radius along its length.

After the radiuses are determined for all segments as described at the block 606, a stent can be selected to have a radius that is equal to a largest radius among the spheres determined at the block 606. As an example for illustration, the diameters of the reconstructed vessel 500 can vary between 3.75 millimeter (mm) and 4.5 mm, and the stent can be selected to have a dimeter of 4.5 mm.

At block 608, the method 600 includes determining a length of a portion of a stent that covers each segment. While on a 2D image a particular length of a stent may be sufficient, that particular length might not be sufficient when the stent is deployed due to foreshortening. Particularly, due to the 3D configuration of the reconstructed vessel 500, the reconstructed vessel 500 might be longer than how it appears in a 2D image. For example, the reconstructed vessel 500 may be tortuous and may have twists and bends in 3D space such that the actual length of the vessel is longer than what appears from the perspective of a 2D image. Further, the stent may be compressed or otherwise change its length upon deployment due to interaction with the vessel. Thus, a longer stent might be needed than what appears in the 2D image. As an example for illustration, a length of a portion of a stent can be 1.5 mm to cover what appears to be a 1 mm segment of the reconstructed vessel 500 once the stent is deployed. This difference can be referred to as “foreshortening.”

The neurovascular module 304 estimates such foreshortening in each segment of the plurality of segments determined at the block 604. The neurovascular module 304 can thus determine a length of the stent that covers each segment of the plurality of segments once deployed within the reconstructed vessel 500.

At block 610, the method 600 includes, once determining of the length in each segment is complete for all segments, determining a final proximal point of the stent when deployed within the reconstructed vessel 500. Particularly, the neurovascular module 304 starts at the center point 504 of the distal opening 502 and incrementally adds the lengths of portions of the stent that cover the respective segments of the reconstructed vessel 500 as determined at the block 608, thus reaching a final proximal point of the stent once the stent is deployed within the reconstructed vessel 500.

At block 612, the method 600 includes determining a final length of the stent as a distance between the center point 504 of the distal opening 502 of the reconstructed vessel 500 and the final proximal point determined at the block 610. Particularly, now that the neurovascular module 304 has determined the final proximal point of the stent once deployed, the neurovascular module 304 can determine the distance between such final proximal point and the center point 504 of the distal opening 502 as determined at the block 404 described above. Such distance is the estimated deployed length of the stent.

Referring back to FIG. 4, the method 400 continues at block 408, where the method 400 includes, once the final length of the stent is determined, estimating radial expansion of the stent within the reconstructed vessel 500. The stent is expected to expand once deployed and released from the catheter into the blood vessel. The neurovascular module 304 is configured to determine the extent of such expansion based on the material of the stent and the length determined at the block 612.

At block 410, the method 400 includes visually presenting the stent deployed within the reconstructed vessel 500. As the neurovascular module 304 has determined the final length of the stent and the expected expansion of the stent once deployed, the neurovascular module 304 can generate a display of the stent deployed within the reconstructed vessel 500 to help the healthcare professional visualize how the position and coverage of the sent once deployed within the vessel of the patient.

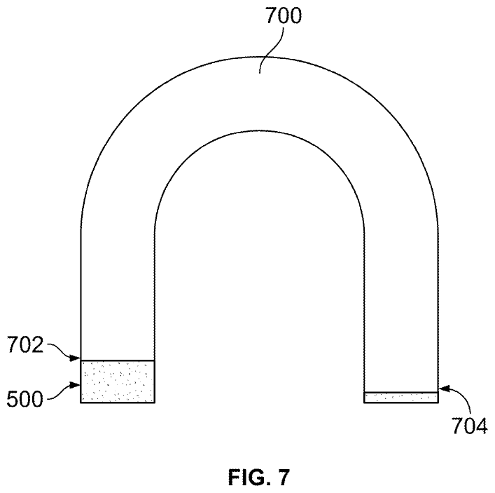

FIG. 7 illustrates a stent 700 deployed within the reconstructed vessel 500, according to an example implementation. As depicted, the visualization of the stent 700 within the reconstructed vessel 500 indicates a starting point 702 and an end point 704 of the stent 700, which takes into consideration foreshortening effect as described above. As mentioned above, the reconstructed vessel 500 represents a simplification of an actual vessel to describe the methods 400, 600. Actual patient vessels might have more complex shapes.

FIG. 8 illustrates a cross-sectional view of a stent 800 deployed within a vessel 802 having an aneurysm 804, according to an example implementation. The vessel 802 has a complex geometry and may represent a realistic vessel of a patient.

The neurovascular module 304 can perform the operations described above with respect to the methods 400, 600 to generate the visualization in FIG. 8 of the stent 800 deployed within the vessel 802 (e.g., a reconstructed vessel) of a patient. The visualization in FIG. 8 depicts the starting point and the end point of the stent 800 once deployed within the vessel 802, and thus shows whether the stent 800 sufficiently covers a neck portion 806 of the aneurysm 804 and whether the stent 800 would be effective in diverting flow away from the aneurysm 804.

Further, referring back to FIG. 4, at block 412, the method 400 includes determining key parameters of the stent 800 of FIG. 8. For example, the of neurovascular module 304 can provide information indicative of vessel wall apposition and pore density variation of the stent 800.

Apposition of the stent 800 may refer to how closely the exterior peripheral surface of the stent 800 interfaces with the inner wall of the vessel 802. If the outer diameter of the stent 800 is smaller than the inner diameter of the vessel 802, the stent 800 can be characterized as having loose appositioning on the wall of the vessel 802. Such loose appositioning might not be desirable as it could lead to migration or movement of the stent 800 once deployed within the vessel 802. It is rather desirable to have the stent 800 with high apposition such that the stent 800 is as close as possible to the wall of the vessel 802 to be stable in its position within the vessel 802 and to provide effective flow diversion.

Apposition can be represented by a coverage percentage at a particular cross section of the vessel 802. For example, at a given cross section of the vessel 802, the coverage percentage (coverage %) can be determined as follows:

$$Coverage \% = \frac{\begin{matrix}Cross Sectional Area of the Vessel - \\ Cross Sectional Area of the Stent\end{matrix}}{Cross Sectional Area of the Vessel}$$

As such, the neurovascular module 304 can determine the coverage percentage at different cross sections along a length of the vessel 802. The neurovascular module 304 can then present the coverage percentage information to the healthcare professional such that the healthcare professional can assess the apposition of the stent 800 once deployed within the vessel 802.

FIG. 9 illustrates a graph 900 showing variation of apposition of the stent 800 along a length of the vessel 802, according to an example implementation. In the graph 900, coverage percentage is shown on the right y-axis, cross sectional area of the stent 800 and the vessel 802 is shown on the left y-axis in millimeters squared (mm²), and a length of the vessel 802 is shown on the x-axis in mm.

Line 902 shows variation of the cross sectional area of the vessel 802 along a length of the vessel 802. As shown at a region 904 corresponding to the location of the aneurysm 804, the cross sectional area of the vessel 802 increases due to the bulging.

Line 906 shows variation of the cross sectional area of the stent 800 along a length of the vessel 802. As shown by the line 906, the cross sectional area of the stent 800 begins (from the left side) with a relatively large cross sectional area then narrows slightly at the region 904 then expands again thereafter toward the end of the vessel 802.

Line 908 shows variation of the coverage percentage or apposition of the stent 800 as determined by the equation above. As shown by the line 908, the coverage percentage starts (from the left side) with a relatively large value (e.g., close to 90%) indicating close interface between the stent 800 and the vessel 802, then decreases at the region 904, then increases again after the region 904 to a value of about 90%. The healthcare professional may assess the apposition information and may decide whether to performance of the stent 800 would be acceptable or whether tweaks to its parameters (e.g., diameter of the stent) could be made to enhance its performance. In examples, rather than providing a graph showing variation in the apposition, the neurovascular module 304 may provide an average apposition value to the healthcare professional.

The neurovascular module 304 may further provide information indicative of porosity (e.g., pore density) of the stent 800. Porosity is the percentage of void space in the stent 800. As mentioned above, the stent 800 being a braided stent has a dense mesh of interwoven wires, which form diamond-shaped holes between the interwoven wires throughout the stent 800. Porosity can be defined as the ratio of the volume of the voids or holes divided by the total volume of the stent 800.

Another way to indicate porosity is the density of such holes (e.g., how many holes are there) at a particular region. Such density may indicate the flow diversion capability of the stent 800 at such region. Too many holes may indicate poor flow diversion as blood can diffuse through the stent 800 into the aneurysm 804, while fewer, and/or smaller holes may indicate enhanced flow diversion capability.

In examples, porosity of the stent 800 may vary along a length of the stent 800 upon deployment into the vessel 802. Such variation may be based on diameter and curvature of the vessel 802. During deployment of the stent 800 within the vessel 802, the stent 800 may be compacted, thereby causing its porosity to decrease in some regions. The neurovascular module 304 is configured to estimate porosities at different regions of the stent 800 based on expected compaction and curvatures using geometric rules. The neurovascular module 304 can present such porosity information to the healthcare professional to assess efficacy of the stent 800 in diverting blood flow away from the aneurysm 804.

In an example, the neurovascular module 304 is configured to visually present the key parameters as well as a visualization of the stent within the vessel via a graphical user interface (GUI) on the display device 306. Such GUI can have different configurations. An example GUI is described next with respect to FIG. 10.

FIG. 10 illustrates a GUI 1000, according to an example implementation. The neurovascular module 304 is configured to generate a display of or visually present the GUI 1000 on the display device 306 to help healthcare professionals visualize a neurovascular device selected for a particular patient with an aneurysm and to provide key parameters of such neurovascular device.

The GUI 1000 can have user-selectable, on-screen graphical items (e.g., buttons, menus, widgets, scroll bars, graphical objects, audio indicators, icons, etc.) to facilitate user-interaction. Particularly, the neurovascular module 304 generates the display of the GUI 1000 on the display device 306, and the healthcare professional can then interact with the GUI 1000 select the user-selectable user-interface items by pressing or selecting areas on a touchscreen of the display device 306, or example.

The GUI 1000 can have a device visualization display area 1002 that shows the type of device that is suitable for a particular vessel and aneurysm as determined by the neurovascular module 304. For example, for a vessel 1004 that is angled and having an aneurysm 1006 at the inflection point of the vessel 1004, the neurovascular module 304 may determine that a stent 1008 (e.g., a braided stent) is suitable. As depicted, the neurovascular module 304 provides a visual representation of the stent 1008 deployed within the vessel 1004 after the operations of the methods 400, 600 are completed.

On the other hand, for another vessel 1010 that is T-shaped, e.g., the vessel 1010 has a straight vessel section 1012 and a branch vessel 1014 that is substantially perpendicular to the straight vessel section 1012. The vessel 1010 has an aneurysm 1016 formed at a junction 1018 between the straight vessel section 1012 and the branch vessel 1014. In this case, the neurovascular module 304 may determine that a coiling device 1020.

In an example, both visualizations (e.g., for the vessel 1004 and the vessel 1010) may be presented simultaneously if they belong to the same patient and both vessels are selected by the healthcare professional (e.g., via vessels accordion 1038 described below). In another example, one vessel is presented at a time.

In addition to the device visualization display area 1002, the GUI 1000 may have a deployment summary display area 1022 providing the key parameters determined by the neurovascular module 304 for the neurovascular device (e.g., stent or coil). As an example, for a braided stent such as the stent 1008, the deployment summary display area 1022 may include several GUI items (e.g., message boxes or information widgets) such as GUI item 1024 providing a final deployed length of the stent, a GUI item 1026 providing a deployed braid angle (e.g., half of the angle made by crossing filaments in the braid of the braided stent), a GUI item 1028 providing average apposition of the stent, and a GUI item 1030 providing a pore density of the stent. More or fewer parameters may be provided.

In an example, the GUI 1000 may have a side bar or side menu 1032 providing several menu items that facilitate interaction between the healthcare professional and the GUI 1000. For example, the side menu 1032 may have a menu item 1034 showing the type of neurovascular device selected and to which the information in the deployment summary display area 1022 pertains.

Further, the side menu 1032 may have several accordions (e.g., a vertically stacked list of items that utilizes show/hide functionality). For example, for a braided stent, the side menu 1032 can have a braided stent accordion 1036 that lists stent configuration or brand/type options. When the “Braided Stent” label is clicked, it expands the section showing the contents within, which include several stent brands/types to be selected. Choosing different brands may cause the display device 306 to alter the information and images displayed in the deployment summary display area 1022 and the device visualization display area 1002, for example.

The side menu 1032 may have a vessels accordion 1038 that, when clicked, may present different vessels of a patient. One or more vessels (e.g., the vessel 1004 and/or the vessel 1010) may then be selected by the healthcare professional to show information of respective neurovascular device pertinent to the selected vessels. The side menu 1032 can have an add-ons section 1040 that can allow the healthcare professional to select information to show in the GUI 1000 in the form of charts or tables.

In some cases, when a stent is deployed in a tortuous vessel, stent “ribboning” may occur. A tortuous vessel can be defined as a complex blood vessel having repeated turns or bends, winding or twisting, etc. Due to such tortuousness, there could be sections of a stent deployed within such vessel that do not expand properly to fill the vessel. Particularly, the stent may twist over itself, or due to expansion and compression multiple times during deployment, the stent may lose its ability to expand. Such non-expansion can be referred to as ribboning, and it reduces the effectiveness of the stent as a flow diverting device.

FIG. 11 illustrates a micro-CT image 1100 of a vessel 1102 having an aneurysm 1104 and a braided stent 1106 exhibiting ribboning. As shown, the vessel 1102 is a tortuous vessel with at least one bend region 1108.

The braided stent 1106 has a ribboned section 1110 at the bend region 1108 where the braided stent 1106 has not expanded to fill the vessel 1102 (poor appositioning). It may be desirable to provide information to the healthcare professional indicating regions of the vessel 1102 where ribboning is most likely to occur. Such foreknowledge may prompt the healthcare professional to use a particular technique or to be cautious in deploying the stent at the bend region 1108 to avoid ribboning where it is most likely to occur.

FIG. 12 illustrates a reconstructed vessel 1200 with an aneurysm 1202 and a region 1204 where ribboning is most likely to occur, according to an example implementation. The neurovascular module 304 may generate the reconstructed vessel 1200 based on micro-CT scan images as described above with respect to FIG. 3. The neurovascular module 304 can then evaluate the geometry and tortuousness of the reconstructed vessel 1200 to determine regions such as the region 1204 where ribboning may occur while deploying a stent 1206.

For example, the neurovascular module 304 may determine sections of the reconstructed vessel 1200 where multiple bends are adjacent to each other, a region where the reconstructed vessel 1200 narrows to a diameter below a threshold diameter compared to respective diameters of adjacent regions, etc. The neurovascular module 304 may use such criteria to determine the regions such as the region 1204 where ribboning is most likely to occur.

The neurovascular module 304 may then generate a display or visually present (e.g., on the GUI 1000) the reconstructed vessel 1200 with or without the stent 1206 shown within the region 1204 labelled as a region where ribboning is most likely to occur. The healthcare professional may then take such information into consideration and adjust the deployment technique to preclude ribboning from occurring at the region 1204.

In some examples, the neurovascular module 304 may further estimate fatigue safety factor for a neurovascular devices, e.g., a stent, considering the apposition, stent materials, foreshortening effects. Knowing the fatigue safety factor may enhance a healthcare professional's confidence post deployment of the device. In an example, the neurovascular module 304 may estimate the fatigue safety factor by leveraging spatial distribution of RGB/grayscale values of a deployed stent in a vessel to quantify mechanical stresses in a heat map, which can further be used to highlight distribution of fatigue factor of safety on each wire of the stent in a given configuration.

As aneurysm progression and rupture is governed by progressive degradation and weakening of the wall of an aneurysm in response to abnormal hemodynamics, it may be desirable to have a tool that helps investigate the relationship between the intra-aneurysmal hemodynamic conditions and wall mechanical properties in aneurysms. This enhances aneurysm evaluation and patient management.

Aneurysm sac characterization for both qualitative and quantitative parameters may include ostium max size, sac area, and sac volume as examples. Such geometric characteristics along with blood flow patterns can be used to predict rupture risk for an aneurysm.

Thus, in some examples, the neurovascular module 304 determines blood flow characteristics in a vessel and aneurysm, and can generate a visualization of blood flow in the vessel and aneurysm after deployment of neurovascular flow diverting device. The neurovascular module 304 can also provide quantitative parameters associated with the blood flow characteristics and performance of a flow diverter (e.g., stent or coil).

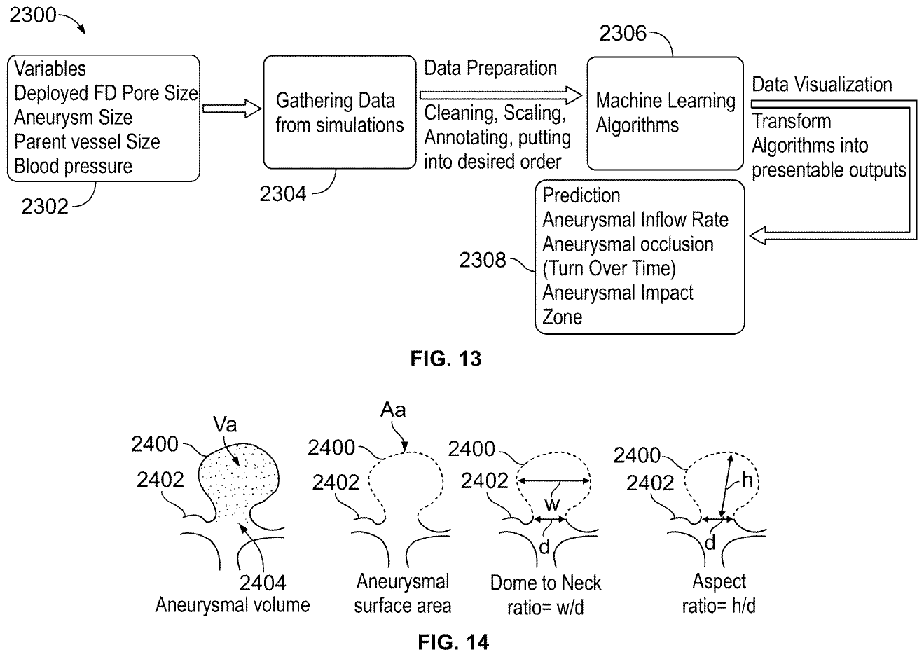

FIG. 13 illustrates a block diagram of a system 2300 for flow visualization and quantification, according to an example implementation. The system 2300 can be implemented by the neurovascular module 304, for example.

At block 2302, the system 2300 receives variables or parameters associated with the flow diverting (FD) device selected to treat the aneurysm, geometric characteristics of the aneurysm and the vessel from which the aneurysm bulges, patient information, etc. For example, the system 2300 can receive or has access to pore size and pore density of the FD device when deployed (e.g., as described above with respect to item 1030 of the GUI 1000). The system 2300 also determines or has access to the aneurysm geometric characteristics/size.

FIG. 14 illustrates different geometric characteristics of an aneurysm 2400 bulging from a vessel 2402, according to an example implementation. Based on the images captured by the image-capture device 302, the system 2300 can determine aneurysm geometric characteristics such as ostium max size, surface area Aa, volume Va, neck ratio, aspect ratio, etc., which can be extracted from the CT images captured by the image-capture device 302, for example.

The system 2300 also determines or receives information indicative of geometry and size of the vessel 2402 from which the aneurysm 2400 bulges. The system 2300 also has access to patient information, such as blood pressure.

Referring back to FIG. 13, at block 2304, the system 2300 gathers date from simulations performed by the neurovascular module 304. As mentioned above, with respect to FIG. 10, for example, such simulations result in a visualization of the deployed FD device in the vessel, staring point and landing zone of the FD device (e.g., stent), deployed length, porosity, etc. The porosity of the FD device can be determined across the aneurysm in 3D.

The system 2300 can further clean, scale, annotate, order, organize the data to provide it to a machine learning (ML) model or algorithm at block 2306. The ML model can be trained with data sets from previous experiences, other patients, etc. Based on such training, the ML model is trained to find a relationship between output performance parameters as described below and characteristics of the aneurysm, vessel, and the selected FD device (the input variables). Particularly, the ML model is configured to use data provided from block 2304 an input data to be processed via the ML model and generate at block 2308 quantitative predictions about performance of the FD device and visualizations of blood flow in the vessel and aneurysm, for example.

This way, the system 2300 (the neurovascular module 304) can provide insights to healthcare professionals to assess the flow diverting capability and efficacy of the devices. As examples, at the block 2308, the neurovascular module 304 can provide parameters such as aneurysm inflow rate, aneurysm occlusion (turnover time), and aneurysmal impact zone, which in turn help healthcare professionals in selecting and deployment of devices.

Aneurysmal inflow can be defined as the average rate of blood flow Q (t) entering the aneurysm (aneurysmal sac) through the a neck-plane 2404 shown in FIG. 14 over the duration of one cardiac cycle T. The neck-plane 2404 can be defined as the plane where the aneurysmal sac (the aneurysm 2400) intersects the parent-vessel (the vessel 2402). The average rate of blood flow Q_{avg} can be determined as follows:

$${Q}_{avg} = \frac{1}{T} {\int }_{0}^{T} Q ( t ) dt$$

The turnover time Tₜ can be defined as the duration of time it takes to fill the aneurysmal sac (e.g., fill the volume Va of the aneurysm). The turnover time can be determined as:

$${T}_{t} = \frac{{V}_{a}}{{Q}_{avg}}$$

where Vₐ is the volume of the aneurysm 2400.

The impact zone (IZ) can be defined as fraction or percentage of the aneurysmal sac surface area Aa on which blood flow impinges. It can be calculated as the total surface area of the aneurysmal sac at peak systole:

$$IZ = \frac{{I}_{a}}{{A}_{a}}$$

where Iₐ is the impact area, and Aₐ is the total surface area.

As such, the system 2300 can provide the healthcare professional with a visualization of blood flow before and after deploying the FD device. In this manner, the healthcare professional can assess performance of the FD device and select the appropriate FD device.

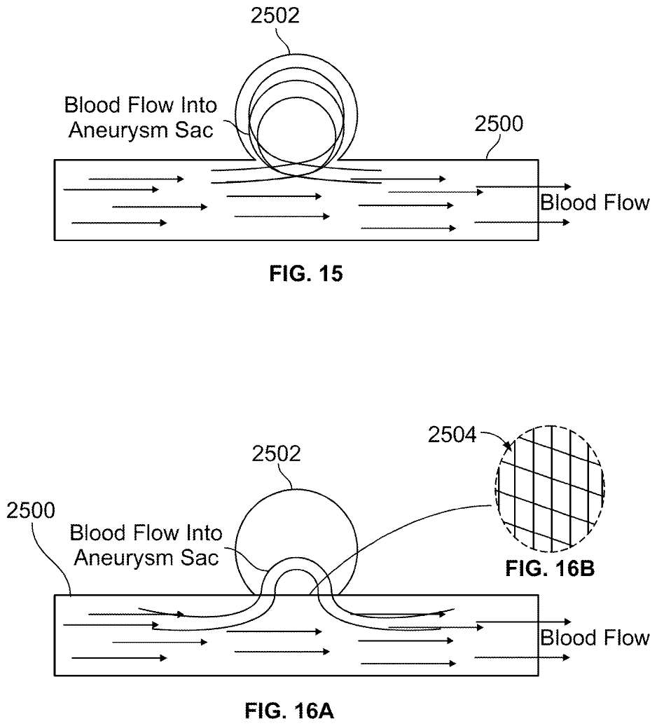

FIG. 15 illustrates blood flow through a vessel 2500 and aneurysm 2502 before treatment (before deploying an FD device), FIG. 16A illustrates blood flow through the vessel 2500 and the aneurysm 2502 after treatment (after deploying an FD device 2504), and FIG. 16B illustrates pore area of the FD device 2504, according to an example implementation. FIGS. 15, 16A depict an example blood flow visualization that the system 2300 (the neurovascular module 304) can provide to a healthcare professional. The FD device 2504 can be a braided stent, for example.

As shown in FIG. 15 blood flows from the vessel 2500 into the aneurysm 2502 and circulates therein, impacting its interior surface. In FIG. 16A, the FD device 2504 substantially suppresses or reduces blood flow into the aneurysm 2502. As such, the FD device 2504 slows blood flow (reduces Q_{avg} and increases Tₜ) within the aneurysm 2502, and can thus enhance the likelihood of forming a clot or thrombosis to protect the aneurysm 2502 from rupturing. The FD device 2504 also mitigates impact (e.g., reduces IZ) of blood flow on the interior surfaces of the aneurysm 2502, thereby reducing the likelihood of rupture.

Thus, for the FD device 2504, the system 2300 provides a visual representation of the predicted performance of the FD device 2504 to help the healthcare professional evaluate the performance and determine adequacy of the FD device 2504 in treating the patient. Additionally, the system 2300 can further provide numerical values indicative of the performance.

The system 230 can thus help in risk mitigation, selection, and sizing of treatment (neurovascular devices) based on these predicted mechanical/geometrical properties of the vessel/Aneurysm. The aneurysm wall mechanical properties can be evaluated for rupture strength, stiffness, and modulus. The geometrical properties include aneurysm volumetric details, dome to neck ratio, aspect ratio, wall thickness and other surface and volumetric details as shown in FIG. 14.

For example, based on such parameters and properties, the system 2300 can provide an indication of changes in aneurysmal inflow rate (e.g., Q(t) or Q_{avg}), aneurysm occlusion, aneurysmal impact zone, which provide an assessment of rupture risk.

Table 1 below provides output parameters that can be provided by the system 2300 as an example.

TABLE 1

| Output Parameter | Untreated | | Treated | | % Improvement |
|---|---|---|---|---|---|
| Aneurysmal Inflow | 0.6 | ml/s | 0.07 | ml/s | 89% |
| Rate | | | | | |
| Aneurysm occlusion | 0.3 | s | 4.32 | s | 93% |
| (turnover time) | | | | | |

| | | | |
|---|---|---|---|
| Aneurysmal Impact | 28.63% | 1.06% | 96.3% |
| Zone | | | |

As indicated by the information in Table 1, the FD device 2504 reduces aneurysmal inflow rate from 0.6 milliliter per second (ml/s) to 0.07 ml/s, which is an 89% percent reduction in flow rate, thereby enhancing the likelihood that a clot may form inside the aneurysm 2502 to protect it from rupture. Aneurysm occlusion is represented by turnover time in seconds indicating that the turnover time increased from 0.3 s to 4.32 s. As such, the turnover time increased by about 93%. Thus, blood flow fills the aneurysm 2502 slowly and spends more time therein (e.g., flow has slowed and blood is not flowing out of the aneurysm 2502 quickly) when the FD device 2504 is deployed. Also, the impact zone has decreased by about 96.3%, indicating a much reduced surface impact area within the aneurysm 2502, and thus enhanced protection against rupture.

As such, the blood flow visualization and performance assessment of a particular FD device can guide the healthcare professional to choose the right device and adjust the treatment options interventionally.

Further, in some examples, the neurovascular module 304 may estimating integrity characteristics of a vessel based on radiodensity, which opacity to the radio wave and X-ray portion of the electromagnetic spectrum: that is, the relative inability of radio and X-ray electromagnetic radiation to pass through a particular material (e.g., the vessel). This way, the neurovascular module 304 can rank the mechanical strength factor of the vessel taking into consideration the radiodensity and age parameters of patients. This feature may help healthcare professionals to choose the right device, such as different stiffness profiles, hardness, braid angles etc., of a stent, or combination of devices as a hybrid setup like stent-coil combo and similar.

The operations performed by the neurovascular module 304 (e.g., the system 2300), such as image processing, training the ML model, processing information through the ML model, visualization, etc. can be computationally intensive and may involve processing a large amount of data. As such, it may be desirable to use a cloud system for storing and processing the data. A cloud system may also facilitate accumulating data from many patients within a healthcare facility or from other facilities to enhance training of ML models, for example.

However, given that it may be desirable to have real-time decisions as a healthcare professional treats a patient, Edge Computing and High Performance Computing (H Computing) techniques can be employed to reduce latency. High Performance Computing generally refers to aggregating computing power in a way that delivers much higher performance than that obtained out of a typical desktop computer or workstation in order to process the large amounts of data and images involved in performing the operations described above.

Edge computing involves having a local cloud system (e.g., at a hospital) where the data is being generated. Rather than transmitting raw data to a central data center for processing and analysis, processing and analysis may instead be performed where the data is generated (e.g., at a hospital or healthcare facility). This may reduce latency and help real-time decision making, wherein data is processed in milliseconds.

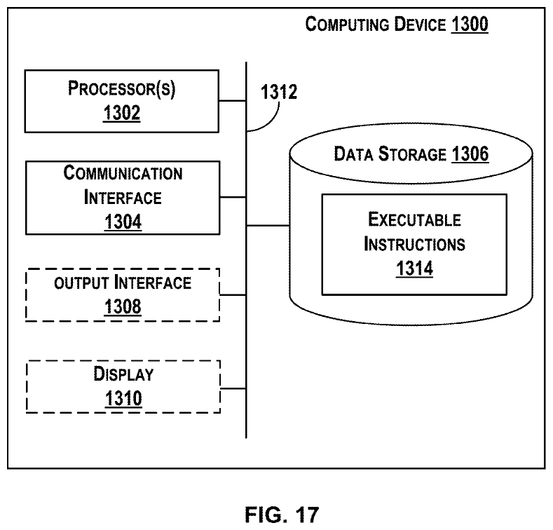

FIG. 17 is a block diagram of a computing device 1300, according to an example implementation. The computing device 1300 can represent, or can be included in, any of the devices described above (e.g., the image-capture device 302, the neurovascular module 304, the display device 306, etc.).

The computing device 1300 may have processor(s) 1302, a communication interface 1304, and data storage 1306, each connected to a communication bus 1312. The computing device 1300 may also include hardware to enable communication within the computing device 1300 and between the computing device 1300 and other devices. The hardware may include transmitters, receivers, and antennas, for example

The communication interface 1304 may be a wireless interface and/or one or more wireline interfaces that allow for both short-range communication and long-range communication to one or more networks or to one or more remote devices (e.g., to allow communication with the communication bus 1312). Such wireless interfaces may provide for communication under one or more wireless communication protocols, Bluetooth, Wi-Fi (e.g., an institute of electrical and electronic engineers (IEEE) 802.11 protocol), Long-Term Evolution (LTE), cellular communications, near-field communication (NFC), and/or other wireless communication protocols. Wireline interfaces may include an Ethernet interface, a CAN network interface, a USB interface, or similar interface to communicate via a wire, a twisted pair of wires, a coaxial cable, an optical link, a fiber-optic link, or other physical connection to a wireline network.

The data storage 1306 may include or take the form of one or more computer-readable storage media that can be read or accessed by the processor(s) 1302. The computer-readable storage media can include volatile and/or non-volatile storage components, such as optical, magnetic, organic or other memory or disc storage, which can be integrated in whole or in part with the processor(s) 1302. The data storage 1306 is considered non-transitory computer-readable media. In some examples, the data storage 1306 can be implemented using a single physical device (e.g., one optical, magnetic, organic or other memory or disc storage unit), while in other examples, the data storage 1306 can be implemented using two or more physical devices.

The data storage 1306 is thus a non-transitory computer readable storage medium, and executable instructions 1314 are stored thereon. The executable instructions 1314 include computer executable code. When the executable instructions 1314 are executed by the processor(s) 1302, the processor(s) 1302 are caused to perform operations of the computing device 1300 (e.g., operations performed by the image-capture device 302, the neurovascular module 304, or the display device 306).

The processor(s) 1302 may be a general-purpose processor or a special purpose processor (e.g., digital signal processors, application-specific integrated circuits (ASIC), etc.). The processor(s) 1302 may receive inputs from the communication interface 1304, and process the inputs to generate outputs that are stored in the data storage 1306. The processor(s) 1302 can be configured to execute the executable instructions 1314 (e.g., computer-readable program instructions) that are stored in the data storage 1306 and are executable to provide the functionality of the computing device 1300 described herein.

If the computing device 1300 represents the display device 306, the computing device 1300 can further include an output interface 1308 and a display 1310. The output interface 1308 outputs information to the display 1310 or to other components as well. Thus, the output interface 1308 can be a wireless interface (e.g., transmitter) or a wired interface as well. The processor(s) 1302 may receive inputs from the communication interface 1304, and process the inputs to generate outputs to the display 1310.

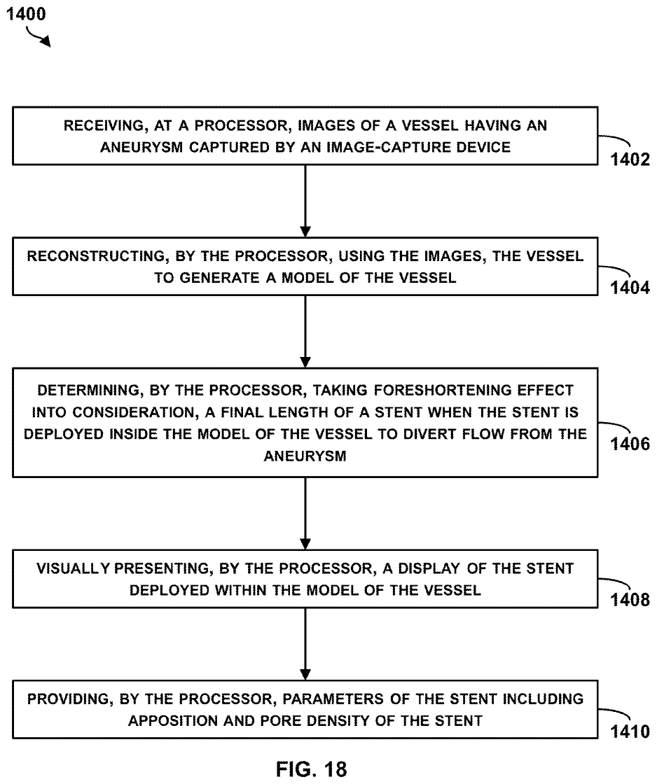

FIG. 18 is a flowchart of a method 1400 for selecting a neurovascular device and providing key parameters of the neurovascular device, according to an example implementation. The method 1400 can, for example, be performed by the neurovascular module 304.

The method 1400 may include one or more operations, or actions as illustrated by one or more of blocks 1402-1410, 1500, 1600-1608, 1700-1702, 1800, 1900, 2000, 2100-2102, 2200-2202. Although the blocks are illustrated in a sequential order, these blocks may in some instances be performed in parallel, and/or in a different order than those described herein. Also, the various blocks may be combined into fewer blocks, divided into additional blocks, and/or removed based upon the desired implementation.

In addition, for the method 1400 and other processes and operations disclosed herein, the flowchart shows operation of one possible implementation of present examples. In this regard, each block may represent a module, a segment, or a portion of program code, which includes one or more instructions executable by processors for implementing specific logical operations or steps in the process. The program code may be stored on any type of computer readable medium or memory, for example, such as a storage device including a disk or hard drive. The computer readable medium may include a non-transitory computer readable medium or memory, for example, such as computer-readable media that stores data for short periods of time like register memory, processor cache and Random Access Memory (RAM). The computer readable medium may also include non-transitory media or memory, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, a tangible storage device, or other article of manufacture, for example. In addition, for the method 1400 and other processes and operations disclosed herein, one or more blocks in FIG. 18 may represent circuitry or digital logic that is arranged to perform the specific logical operations in the process.

At block 1402, the method 1400 includes receiving, at a processor (e.g., the processor(s) 1302 of the neurovascular module 304), images of a vessel having an aneurysm captured by the image-capture device 302.

At block 1404, the method 1400 includes reconstructing, by the processor, using the images, the vessel to generate a model of the vessel (e.g., a 3D model of the vessel such as the reconstructed vessel 500, the vessel 802, or the vessel 1004).

At block 1406, the method 1400 includes determining, by the processor, taking foreshortening effect into consideration, a final length of a stent when the stent is deployed inside the model of the vessel to divert flow from the aneurysm. Determining the final length of a stent is described above with respect to the block 404 and the method 600.

At block 1408, the method 1400 includes visually presenting (e.g., on the display device 306), by the processor, a display of the stent deployed within the model of the vessel. Such visual presentation is shown in FIG. 10, for example.

At block 1410, the method 1400 includes providing, by the processor, parameters of the stent including apposition and pore density of the stent. For example, the parameters can be displayed as shown in FIG. 10.

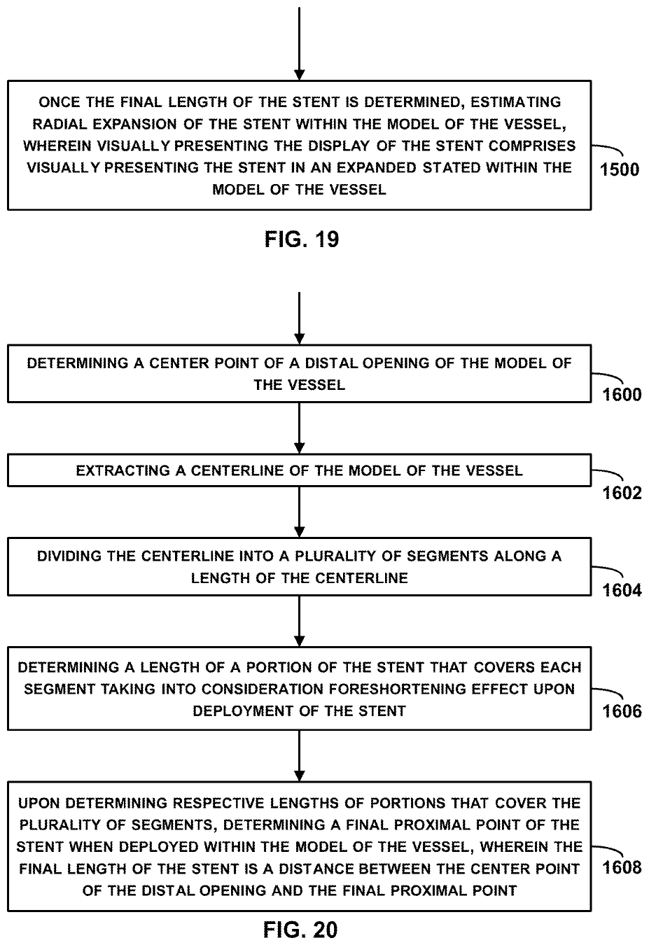

FIG. 19 is a flowchart of additional operations that are executable with the method 1400, according to an example implementation. At block 1500, the operations include, once the final length of the stent is determined, estimating radial expansion of the stent within the model of the vessel, wherein visually presenting the display of the stent comprises visually presenting the stent in an expanded stated within the model of the vessel. This is described above with respect to block 408 of the method 400, for example.

FIG. 20 is a flowchart of additional operations that are executable with the method 1400, according to an example implementation. Determining, taking foreshortening effect into consideration, the final length of the stent involves several operations. At block 1600, the operations include determining a center point (e.g., the center point 504) of a distal opening (e.g., the distal opening 502) of the model of the vessel (e.g., the model of the reconstructed vessel 500). At block 1602, the operations include extracting a centerline (e.g., the centerline 506) of the model of the vessel.

At block 1604, the operations include dividing the centerline into a plurality of segments along a length of the centerline. At block 1606, the operations include determining a length of a portion of the stent that covers each segment taking into consideration foreshortening effect upon deployment of the stent. At block 1608, the operations include, upon determining respective lengths of portions that cover the plurality of segments, determining a final proximal point of the stent when deployed within the model of the vessel, wherein the final length of the stent is a distance between the center point of the distal opening and the final proximal point.

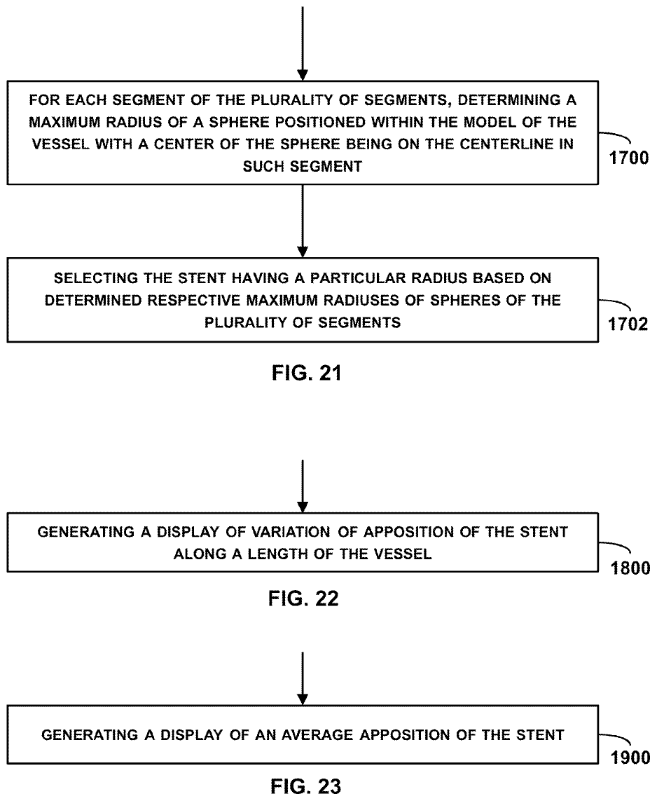

FIG. 21 is a flowchart of additional operations that are executable with the method 1400, according to an example implementation. At block 1700, the operations include, for each segment of the plurality of segments, determining a maximum radius of a sphere positioned within the model of the vessel with a center of the sphere being on the centerline in such segment. At block 1702, the operations include selecting the stent having a particular radius based on determined respective maximum radiuses of spheres of the plurality of segments.

FIG. 22 is a flowchart of additional operations that are executable with the method 1400, according to an example implementation. At block 1800, the operations include generating a display of variation of apposition of the stent along a length of the vessel (see FIG. 9).

FIG. 23 is a flowchart of additional operations that are executable with the method 1400, according to an example implementation. At block 1900, the operations include generating a display of an average apposition of the stent (see the GUI item 1028 in FIG. 10).

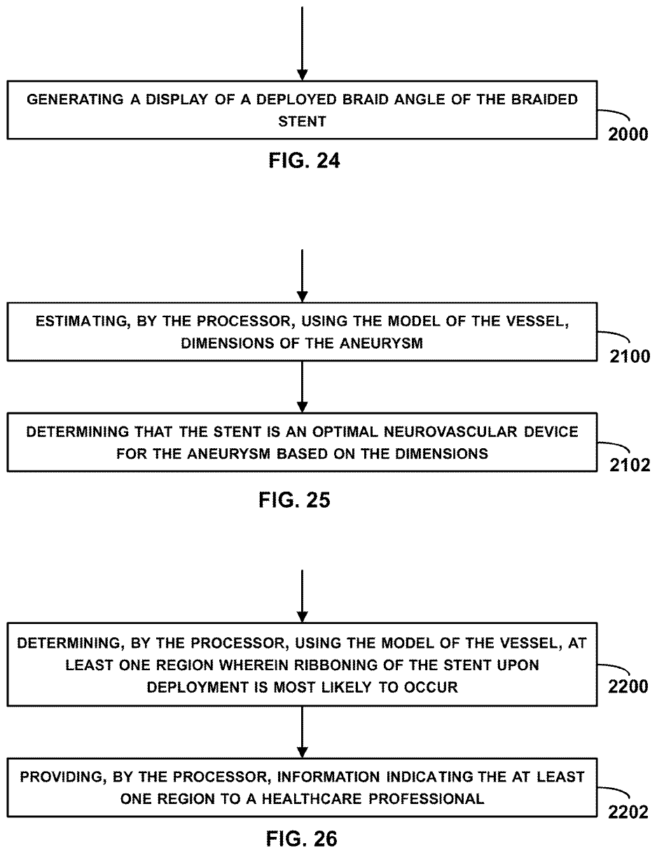

FIG. 24 is a flowchart of additional operations that are executable with the method 1400, according to an example implementation. At block 2000, the operations include generating a display of a deployed braid angle of the braided stent (see the GUI item 1026 in FIG. 10).

FIG. 25 is a flowchart of additional operations that are executable with the method 1400, according to an example implementation. At block 2100, the operations include estimating, by the processor, using the model of the vessel, dimensions (e.g., H, W1, W2, and ratios described with respect to FIG. 2) of the aneurysm. At block 2102, the operations include determining that the stent is an optimal neurovascular device for the aneurysm based on the dimensions. For example, the neurovascular module 304 determines that the vessel is similar to the vessel 1004 rather than the vessel 1010, and therefore determines that a stent is more appropriate for diverting blood flow.

FIG. 26 is a flowchart of additional operations that are executable with the method 1400, according to an example implementation. At block 2200, the operations include determining, by the processor, using the model of the vessel, at least one region wherein ribboning of the stent upon deployment is most likely to occur (see FIGS. 11-12). At block 2202 the operations include providing, by the processor, information indicating the at least one region (e.g., the region 1204) to a healthcare professional.

The detailed description above describes various features and operations of the disclosed systems with reference to the accompanying figures. The illustrative implementations described herein are not meant to be limiting. Certain aspects of the disclosed systems can be arranged and combined in a wide variety of different configurations, all of which are contemplated herein.

Further, unless context suggests otherwise, the features illustrated in each of the figures may be used in combination with one another. Thus, the figures should be generally viewed as component aspects of one or more overall implementations, with the understanding that not all illustrated features are necessary for each implementation.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

Further, devices or systems may be used or configured to perform functions presented in the figures. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner.

By the term “substantially” or “about” it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide.

The arrangements described herein are for purposes of example only. As such, those skilled in the art will appreciate that other arrangements and other elements (e.g., machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. Also, the terminology used herein is for the purpose of describing particular implementations only, and is not intended to be limiting.

Embodiments of the present disclosure can thus relate to one of the enumerated example embodiment (EEEs) listed below.

EEE 1 is a method comprising: receiving, at a processor, images of a vessel having an aneurysm captured by an image-capture device; reconstructing, by the processor, using the images, the vessel to generate a model of the vessel; determining, by the processor, taking foreshortening effect into consideration, a final length of a stent when the stent is deployed inside the model of the vessel to divert flow from the aneurysm; visually presenting, by the processor, a display of the stent deployed within the model of the vessel; and providing, by the processor, parameters of the stent including apposition and pore density of the stent.

EEE 2 is the method of EEE 1, further comprising: once the final length of the stent is determined, estimating radial expansion of the stent within the model of the vessel, wherein visually presenting the display of the stent comprises visually presenting the stent in an expanded stated within the model of the vessel.

EEE 3 is the method of any of EEEs 1-2, wherein determining, taking foreshortening effect into consideration, the final length of the stent comprises: determining a center point of a distal opening of the model of the vessel; extracting a centerline of the model of the vessel; dividing the centerline into a plurality of segments along a length of the centerline; determining a length of a portion of the stent that covers each segment taking into consideration foreshortening effect upon deployment of the stent; and upon determining respective lengths of portions that cover the plurality of segments, determining a final proximal point of the stent when deployed within the model of the vessel, wherein the final length of the stent is a distance between the center point of the distal opening and the final proximal point.

EEE 4 is the method of EEE 3, further comprising: for each segment of the plurality of segments, determining a maximum radius of a sphere positioned within the model of the vessel with a center of the sphere being on the centerline in such segment; and selecting the stent having a particular radius based on determined respective maximum radiuses of spheres of the plurality of segments.

EEE 5 is the method of any of EEEs 1-4, wherein providing the parameters of the stent comprises: generating a display of variation of apposition of the stent along a length of the vessel.

EEE 6 is the method of any of EEEs 1-5, wherein providing the parameters of the stent comprises: generating a display of an average apposition of the stent.

EEE 7 is the method of any of EEEs 1-6, wherein the stent is a braided stent, wherein providing the parameters of the stent comprises: generating a display of a deployed braid angle of the braided stent.

EEE 8 is the method of any of EEEs 1-7, further comprising: estimating, by the processor, using the model of the vessel, dimensions of the aneurysm; and determining that the stent is an optimal neurovascular device for the aneurysm based on the dimensions.

EEE 9 is the method of any of EEEs 1-8, further comprising: determining, by the processor, using the model of the vessel, at least one region wherein ribboning of the stent upon deployment is most likely to occur; and providing, by the processor, information indicating the at least one region to a healthcare professional.

The method of any of EEEs 1-9 can further include any of the operations performed by the neurovascular module of any of EEEs 18-25 below.

EEE 10 is a non-transitory computer-readable medium having stored therein a plurality of executable instructions that, when executed by a processor of a neurovascular module, causes the neurovascular module to perform operations comprising: receiving images of a vessel having an aneurysm captured by an image-capture device; generating, using the images, a model of the vessel; determining, taking foreshortening effect into consideration, a final length of a stent when the stent is deployed inside the model of the vessel to divert flow from the aneurysm; visually presenting a display of the stent deployed within the model of the vessel; and providing parameters of the stent including apposition and pore density of the stent.

EEE 11 is the non-transitory computer-readable medium of EEE 10, wherein the operations further comprise: once the final length of the stent is determined, estimating radial expansion of the stent within the model of the vessel, wherein visually presenting the display of the stent comprises visually presenting the stent in an expanded stated within the model of the vessel.

EEE 12 is the non-transitory computer-readable medium of any of EEEs 10-11, wherein determining, taking foreshortening effect into consideration, the final length of the stent comprises: determining a center point of a distal opening of the model of the vessel; extracting a centerline of the model of the vessel; dividing the centerline into a plurality of segments along a length of the centerline determining a length of a portion of the stent that covers each segment taking into consideration foreshortening effect upon deployment of the stent; and, upon determining respective lengths of portions that cover the plurality of segments, determining a final proximal point of the stent when deployed within the model of the vessel, wherein the final length of the stent is a distance between the center point of the distal opening and the final proximal point.

EEE 13 is the non-transitory computer-readable medium of EEE 12, wherein providing the parameters of the stent comprises: generating a display of variation of apposition of the stent along a length of the vessel.

EEE 14 is the non-transitory computer-readable medium of any of EEEs 10-13, wherein providing the parameters of the stent comprises: generating a display of an average apposition of the stent.

EEE 15 is the non-transitory computer-readable medium of any of EEEs 10-14, wherein the stent is a braided stent, wherein providing the parameters of the stent comprises: generating a display of a deployed braid angle of the braided stent.

EEE 16 is the non-transitory computer-readable medium of any of EEEs 10-15, wherein the operations further comprise: estimating, using the model of the vessel, dimensions of the aneurysm; and determining that the stent is an optimal neurovascular device for the aneurysm based on the dimensions.

EEE 17 is the non-transitory computer-readable medium of any of EEEs 10-16, wherein the operations further comprise: determining using the model of the vessel, at least one region wherein ribboning of the stent upon deployment is most likely to occur; and providing information indicating the at least one region to a healthcare professional.

The non-transitory computer-readable medium of any of EEEs 10-16 can further perform any of the operations performed by the neurovascular module of EEEs 18-25 below.

EEE 18 is a system comprising: an image-capture device configured to capture micro computerized tomography (micro-CT) images of a vessel having an aneurysm; a neurovascular module in communication with the image-capture device, wherein the neurovascular module is configured to perform operations comprising: (i) reconstructing, using the micro-CT images, the vessel to generate a model of the vessel, and (ii) determining, taking foreshortening effect into consideration, a final length of a stent when the stent is deployed inside the model of the vessel to divert flow from the aneurysm; and a display device in communication with the neurovascular module, wherein the display device is configured to perform operations comprising (i) visually presenting a display of the stent deployed within the model of the vessel, and (ii) generating a display of parameters of the stent including apposition and pore density of the stent.

EEE 19 is the system of EEE 18, wherein determining, by the neurovascular module, the final length of the stent comprises: determining a center point of a distal opening of the model of the vessel; extracting a centerline of the model of the vessel; dividing the centerline into a plurality of segments along a length of the centerline; determining a length of a portion of the stent that covers each segment taking into consideration foreshortening effect upon deployment of the stent; and upon determining respective lengths of portions that cover the plurality of segments, determining a final proximal point of the stent when deployed within the model of the vessel, wherein the final length of the stent is a distance between the center point of the distal opening and the final proximal point.

EEE 20 is the system of any of EEEs 18-19, wherein the neurovascular module is further configured to perform operations comprising determining using the model of the vessel, at least one region wherein ribboning of the stent upon deployment is most likely to occur, and providing information indicating the at least one region to a healthcare professional.

EEE 21 is the system of any of EEEs 18-20, wherein the neurovascular module is further configured to perform operations comprising: generating a visualization of blood flow through the vessel and the aneurysm.

EEE 22 is the system of EEE 21, wherein the neurovascular module is further configured to perform operations comprising: generating the visualization of blood flow through the vessel and the aneurysm before deployment of the stent and after deployment of the stent.

EEE 23 is the system of any of EEEs 18-22, wherein the neurovascular module is further configured to perform operations comprising: providing information indicative of aneurysmal inflow rate, aneurysmal occlusion or turnover time, and aneurysmal impact zone after deployment of the stent.

EEE 24 is the system of any of EEEs 18-23, wherein the neurovascular module is further configured to perform operations comprising: estimating a fatigue safety factor for the stent taking into consideration the apposition, stent materials, and the foreshortening effect.

EEE 25 is the system of any of EEEs 18-24, wherein the neurovascular module is further configured to perform operations comprising: estimating one or more vessel integrity characteristics based on radiodensity and one or more age parameters of a patient.

What is claimed is:

1. A method comprising:
receiving, at a processor, images of a vessel having an aneurysm captured by an image-capture device;
reconstructing, by the processor, using the images, the vessel to generate a model of the vessel;
determining, by the processor, taking foreshortening effect into consideration, a final length of a stent when the stent is deployed inside the model of the vessel to divert flow from the aneurysm;
visually presenting, by the processor, a display of the stent deployed within the model of the vessel; and
providing, by the processor, parameters of the stent including apposition and pore density of the stent.

2. The method of claim 1, further comprising:
once the final length of the stent is determined, estimating radial expansion of the stent within the model of the vessel, wherein visually presenting the display of the stent comprises visually presenting the stent in an expanded stated within the model of the vessel.

3. The method of claim 1, wherein determining, taking foreshortening effect into consideration, the final length of the stent comprises:
determining a center point of a distal opening of the model of the vessel;
extracting a centerline of the model of the vessel;
dividing the centerline into a plurality of segments along a length of the centerline;
determining a length of a portion of the stent that covers each segment taking into consideration foreshortening effect upon deployment of the stent; and
upon determining respective lengths of portions that cover the plurality of segments, determining a final proximal point of the stent when deployed within the model of the vessel, wherein the final length of the stent is a distance between the center point of the distal opening and the final proximal point.

4. The method of claim 3, further comprising:
for each segment of the plurality of segments, determining a maximum radius of a sphere positioned within the model of the vessel with a center of the sphere being on the centerline in such segment; and
selecting the stent having a particular radius based on determined respective maximum radiuses of spheres of the plurality of segments.

5. The method of claim 1, wherein providing the parameters of the stent comprises:
generating a display of variation of apposition of the stent along a length of the vessel.

6. The method of claim 1, wherein providing the parameters of the stent comprises:
generating a display of an average apposition of the stent.

7. The method of claim 1, wherein the stent is a braided stent, wherein providing the parameters of the stent comprises:
generating a display of a deployed braid angle of the braided stent.

8. The method of claim 1, further comprising:
estimating, by the processor, using the model of the vessel, dimensions of the aneurysm; and
determining that the stent is an optimal neurovascular device for the aneurysm based on the dimensions.

9. The method of claim 1, further comprising:
determining, by the processor, using the model of the vessel, at least one region wherein ribboning of the stent upon deployment is most likely to occur; and
providing, by the processor, information indicating the at least one region to a healthcare professional.

10. A non-transitory computer-readable medium having stored therein a plurality of executable instructions that, when executed by a processor of a neurovascular module, causes the neurovascular module to perform operations comprising:
receiving images of a vessel having an aneurysm captured by an image-capture device;
generating, using the images, a model of the vessel;
determining, taking foreshortening effect into consideration, a final length of a stent when the stent is deployed inside the model of the vessel to divert flow from the aneurysm;
visually presenting a display of the stent deployed within the model of the vessel; and
providing parameters of the stent including apposition and pore density of the stent.

11. The non-transitory computer-readable medium of claim 10, wherein the operations further comprise:
once the final length of the stent is determined, estimating radial expansion of the stent within the model of the vessel, wherein visually presenting the display of the stent comprises visually presenting the stent in an expanded stated within the model of the vessel.

12. The non-transitory computer-readable medium of claim 10, wherein
determining, taking foreshortening effect into consideration, the final length of the stent comprises:
determining a center point of a distal opening of the model of the vessel;
extracting a centerline of the model of the vessel;
dividing the centerline into a plurality of segments along a length of the centerline
determining a length of a portion of the stent that covers each segment taking into consideration foreshortening effect upon deployment of the stent; and
upon determining respective lengths of portions that cover the plurality of segments, determining a final proximal point of the stent when deployed within the model of the vessel, wherein the final length of the stent is a distance between the center point of the distal opening and the final proximal point.

13. The non-transitory computer-readable medium of claim 12, wherein providing the parameters of the stent comprises:
generating a display of variation of apposition of the stent along a length of the vessel.

14. The non-transitory computer-readable medium of claim 10, wherein providing the parameters of the stent comprises:
generating a display of an average apposition of the stent.

15. The non-transitory computer-readable medium of claim 10, wherein the stent is a braided stent, wherein providing the parameters of the stent comprises:
generating a display of a deployed braid angle of the braided stent.

16. The non-transitory computer-readable medium of claim 10, wherein the operations further comprise:
estimating, using the model of the vessel, dimensions of the aneurysm; and
determining that the stent is an optimal neurovascular device for the aneurysm based on the dimensions.

17. The non-transitory computer-readable medium of claim 10, wherein the operations further comprise:
determining using the model of the vessel, at least one region wherein ribboning of the stent upon deployment is most likely to occur; and
providing information indicating the at least one region to a healthcare professional.

18. A system comprising:
an image-capture device configured to capture micro computerized tomography (micro-CT) images of a vessel having an aneurysm;
a neurovascular module in communication with the image-capture device, wherein the neurovascular module is configured to perform operations comprising: (i) reconstructing, using the micro-CT images, the vessel to generate a model of the vessel, and (ii) determining, taking foreshortening effect into consideration, a final length of a stent when the stent is deployed inside the model of the vessel to divert flow from the aneurysm; and
a display device in communication with the neurovascular module, wherein the display device is configured to perform operations comprising (i) visually presenting a display of the stent deployed within the model of the vessel, and (ii) generating a display of parameters of the stent including apposition and pore density of the stent.

19. The system of claim 18, wherein determining, by the neurovascular module, the final length of the stent comprises:
determining a center point of a distal opening of the model of the vessel;
extracting a centerline of the model of the vessel;
dividing the centerline into a plurality of segments along a length of the centerline
determining a length of a portion of the stent that covers each segment taking into consideration foreshortening effect upon deployment of the stent; and
upon determining respective lengths of portions that cover the plurality of segments, determining a final proximal point of the stent when deployed within the model of the vessel, wherein the final length of the stent is a distance between the center point of the distal opening and the final proximal point.

20. The system of claim 18, wherein the neurovascular module is further configured to perform operations comprising determining using the model of the vessel, at least one region wherein ribboning of the stent upon deployment is most likely to occur, and providing information indicating the at least one region to a healthcare professional.

21. The system of claim 18, wherein the neurovascular module is further configured to perform operations comprising:
generating a visualization of blood flow through the vessel and the aneurysm.

22. The system of claim 21, wherein the neurovascular module is further configured to perform operations comprising:
generating the visualization of blood flow through the vessel and the aneurysm before deployment of the stent and after deployment of the stent.

23. The system of claim 18, wherein the neurovascular module is further configured to perform operations comprising:
providing information indicative of aneurysmal inflow rate, aneurysmal occlusion or turnover time, and aneurysmal impact zone after deployment of the stent.

24. The system of claim 18, wherein the neurovascular module is further configured to perform operations comprising:
estimating a fatigue safety factor for the stent taking into consideration the apposition, stent materials, and the foreshortening effect.

25. The system of claim 18, wherein the neurovascular module is further configured to perform operations comprising:
estimating one or more vessel integrity characteristics based on radiodensity and one or more age parameters of a patient.
